(19) United States
(12) Patent Application Publication
(10) Pub. No.: US 2023/0260646 A1
(43) Pub. Date: Aug. 17, 2023

(54) RELATIONSHIP ESTIMATATION SYSTEM

(71) Applicants: KYOTO UNIVERSITY, Kyoto (JP); VELDT INC., Tokyo (JP)

(72) Inventors: Ryoichi SHINKUMA, Kyoto (JP); Jin NONOGAMI, Tokyo (JP)

(73) Assignees: KYOTO UNIVERSITY, Kyoto (JP); VELDT INC., Tokyo (JP)

(21) Appl. No.: 18/014,279

(22) PCT Filed: Jun. 17, 2021
(86) PCT No.: PCT/JP2021/023005

§ 371 Date: Jan. 3, 2023

(30) Foreign Application Priority Data
Jul. 3, 2020 (JP) 2020-115945

(51) Int. Cl. G16H 40/67; G06F 16/28

(52) U.S. Cl. CPC G16H 40/67; G06F 16/288

(57) ABSTRACT

The present invention aims to provide a relationship estimation system capable of estimating a relationship of attributes meaningful to a person based on time-series data of attributes relating to a body or activities of a person. The relationship estimation system collects time-series data of attributes relating to a body or activity of a person, calculates similarity between time-series data of the attributes, and generates a relationship graph G in which the attributes are served as nodes, the similarity is served as a link weight, and the nodes are connected by the links. An inter-node having a large total link weight of the inter-node in the relationship graph G is extracted, and the relationship between attribute corresponding to the inter-node is estimated.

### TECHNICAL FIELD

The present invention relates to a relationship estimation system for estimating a relationship of attributes meaningful to a person based on time-series data of the attributes relating to a body or activities of a person.

### BACKGROUND ART

In recent years, in accordance with the need for physical condition management for pandemic countermeasures and a health boom, attention has been paid to the use of state changes within a body of a person in assisting health and beauty maintenance by collecting and visualizing the state changes within the body of the person.

In particular, recently, various wearable devices, such as, e.g., an eyeglass-type device, a wristwatch-type device, and a wristband-type device, to be worn on a person's body have appeared. Applications for such devices are also provided. The applications are configured to collect state changes within a body of a person by sensors mounted on the device and visualize the state changes by merely wearing the device in daily life by the device itself or via a smartphone.

As described above, a smartphone has become available to work with such a wearable device to manage health information and beauty information on the smartphone. However, the data management that can be performed on the smartphone is limited to, for example, information on the step count only, information on the heart rate only, information on the blood pressure only, and the like, and total data management has not been popular.

Under the circumstance, as a system for centrally managing activity information on a body that can be acquired from a plurality of wearable devices, a healthcare system, such as, e.g., “Google Fit (registered trademark)” prepared for an Android terminal and “Healthcare” prepared for an iOS terminal, is provided.

For example, “Google Fit” can collectively store and refer to the information on fitness data provided by Google LLC across devices and application users. In addition, even in the case of “Google Fit” alone, it is possible to record activity information, position information data, body measured values, nutritional information, and sleeping information by using position information of the Android terminal and various sensors. On the other hand, “Healthcare” can record activity information, vital information, body measured values, reproductive health information, test results, nutritional information, mindfulness, sleep information, and the like, from location information of the terminal and various sensors and can read and store the information recorded in “Healthcare” from a third-party application.

### PRIOR ART DOCUMENT

### Non-Patent Document

Non-Patent Document 1: Biofeedback Studies, Volume 44, Issue 2, 2017, “Present Status and Problems of Systems utilizing Wearable Devices, and Future Prospects.”

### SUMMARY OF THE INVENTION

### Problems to be Solved by the Invention

However, although a large number of wearable devices and information management systems have been provided as described above, they have not yet become widespread. The non-popularization reasons are considered as follows. That is, there are many people who do not feel the necessity of wearing expensive wearable devices every day for healthcare purposes. In addition, security aspects, such as, e.g., privacy protection, are not sufficiently secured. Furthermore, it has not been established how to extract meaningful data from a vast number of data accumulated daily. In particular, if data meaningful to a person can be extracted, the importance of using data relating to healthcare and beauty will increase more and more.

The present invention has been made in view of the above-described problems. An object of the present invention is to provide a relationship estimation system capable of estimating a relationship of attributes meaningful to a person, based on time-series data of attributes relating to a body or activities of a person.

### Means for Solving the Problems

In order to attain the above-described objects, a relationship estimation system according to the present invention includes:

a data acquisition unit configured to acquire time-series data of a plurality of attributes, the time-series data including at least time-series data of attributes relating to a body or activities of a person;

a time-series data processing unit configured to calculate similarity between the time-series data of the attributes acquired by the data acquisition unit;

a relationship graph generation unit configured to generate a relationship graph in which the attributes of the time-series data acquired by the data acquisition unit serve as nodes and the nodes are connected by links, the relationship graph generation unit being configured to utilize the similarity between the time-series data of the attributes calculated by the time-series data processing unit to calculate a link weight of an inter-node;

a node data processing unit configured to extract the inter-node having a large total link weight in the relationship graph generated by the relationship graph generation unit; and

a relationship estimation unit configured to estimate a relationship between the attributes corresponding to the inter-node extracted by the node data processing unit.

The relationship graph generation unit may generate the relationship graph by using the similarity between the time-series data of the attributes calculated by the time-series data processing unit as the link weight of the inter-node.

Further, the node data processing unit preferably calculates a plurality of paths having a large link weight for each inter-node in the relationship graph generated by the relationship graph generation unit and extract the inter-node having a large total link weight based on the link weight of each path.

The node data processing unit preferably calculates a total link weight of the inter-node by computing the link weight for each path of the inter-node.

The node data processing unit preferably calculates the total link weight of the inter-node by adding an inverse of a link length for each path of the inter-node as a link weight.

The relationship estimation system preferably further includes:
a causal relationship processing unit configured to assess a causal relationship of the inter-node in the relationship graph by calculating a correlation between the time-series data of the attributes.

The causal relationship processing unit preferably calculates positive and negative polarities and a time gap of the correlation between the time-series data of the attributes.

The node data processing unit preferably orients a link of the inter-node in the relationship graph based on the causal relationship of the inter-node assessed by the causal relationship processing unit.

The relationship estimation unit preferably estimates the relationship between the attributes corresponding to the inter-node, based on the causal relationship of the inter-node assessed by the causal relationship processing unit.

The data acquisition unit preferably acquires the time-series data including at least one of time-series data of attributes measured by a sensor of an external IoT device, time-series data of attributes of an external Web service, and a time-series data of attributes measured or input by an information terminal device mounting the relationship estimation system.

The time-series data processing unit preferably normalizes the time-series data of each of the attributes acquired by the data acquisition unit.

The present invention further provides an information terminal device equipped with the relationship estimation system described above.

The present invention further provides a relationship estimation system including:
the above-described plurality of information terminal devices; and
a server device connected to each of the information terminal devices via a network,
wherein the server device generates a relationship graph based on time-series data of attributes collected from each of the information terminal devices.

### Effects of the Invention

According to the present invention, time-series data of attributes relating to a body or activities of a person are collected. Similarity between the time-series data of the attributes is calculated. A relationship graph is generated in which the attributes serve as nodes, the similarity serves as a link weight, and the nodes are connected by links. Thereafter, an inter-node having a large total link weight of an inter-node in the relationship graph is extracted, and the relationship between attributes corresponding to the inter-node is estimated. For this reason, an inter-node having a large total link weight is extracted while considering comprehensive link paths including not only a direct link path with the similarity calculated but also an indirect link path via other nodes. This makes it possible to accurately estimate a relationship between attributes meaningful to a person.

### BRIEF DESCRIPTION OF THE DRAWINGS

FIG. 1 is a diagram showing an entire configuration of a relationship estimation system.

FIG. 2 is a block diagram showing a configuration of an information terminal device of FIG. 1.

FIG. 3 is a table showing types of time-series data of attributes.

FIG. 4 is a diagram showing a screen example of time-series data of attributes input to an information terminal device.

FIG. 5 is a diagram showing a configuration of a relationship graph in which nodes are connected by links.

FIG. 6 is a table showing nodes, distances of inter-nodes, and paths of a relationship graph.

FIG. 7 is a table showing correlations and time gaps of time-series data of various attributes.

FIG. 8 shows graphs used to calculate a correlation and a time gap of time-series data of two attributes.

FIG. 9 is a diagram showing a screen example of outputting relationships between attributes.

FIG. 10 is a flowchart showing an operation of a relationship estimation system.

FIG. 11 is a diagram showing an outline of a relationship estimation system according to Example 1.

FIG. 12 is a diagram showing an outline of a relationship estimation system according to Example 2.

FIG. 13 is a diagram showing an outline of a relationship estimation system according to Example 3.

### EMBODIMENTS FOR CARRYING OUT THE INVENTION

Next, some embodiments of a relationship estimation system (hereinafter referred to as “this system”) according to the present invention will be described with reference to the attached drawings.

[Overall Configuration of this System]

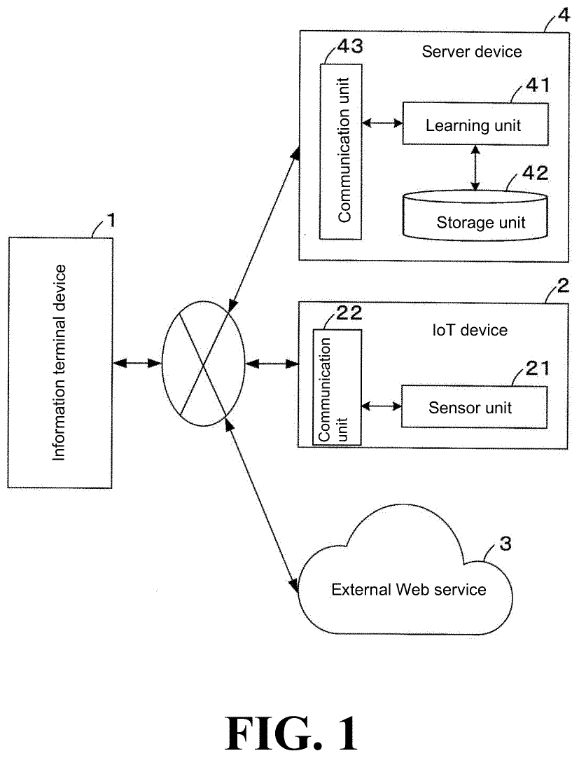

As shown in FIG. 1, this system is provided with a user information terminal device 1, such as, e.g., a smartphone and a robot, an IoT device 2 for measuring time-series data of various attributes, an external Web service 3 for providing time-series data of various attributes, and a server device 4 for collecting time-series data of attributions from each information terminal device 1. The information terminal device 1, the server device 4, the IoT device 2, and the external Web service 3 are connected to each other via a network, such as, e.g., the Internet.

The IoT device 2 is a device for measuring time-series data (healthcare data, life-logs) of one or a plurality of types of attributes relating a body and/or activities of a user (person). The IoT device 2 is provided with a sensor unit 21 for measuring time-series data of each attribute, and a communication unit 22 for transmitting the time-series data of each attribute measured by the sensor unit 21 to the information terminal device 1.

Examples of the IoT device 2 include a wearable device worn by a user, such as, e.g., an eyeglass-type device, a wristwatch-type device, and a wrist-type device. Examples of the sensor include a two-dimensional image sensor, such as, e.g., a camera, a three-dimensional image sensor, such as, e.g., an LIDAR (Light Detection and Ranging), an acceleration sensor, and any sensor that measures time-series data of various attributes. The types of time-series data of attributes measured by the IoT device 2 will be described in the description of the configuration of the information terminal device 1, which will be described later.

The external Web service 3 collects and provides time-series data of one or a plurality of types of attributes mainly relating to natural, social, or economic events, such as, e.g., climates, environments, and locations. The external Web service 3 transmits the collected time-series data of attributes to the information terminal device 1. Examples of the external Web service 3 include services for providing a weather database, an ocean database, a traffic database, and a stock database. The types of the time-series data of attributes provided by the external Web service 3 will be described in the description of the configuration of the information terminal device 1, which will be described later.

The server device 4 receives time-series data of various types of attributes other than personal data from information terminal devices 1 of a large number of users and generates an intensive relationship graph. The server device 4 is provided with a learning unit 41 that generates a relationship graph (model) based on various types of time-series data of a large number of users, a storage unit 42 that stores the relationship graph (model) and time-series data, and the communication unit 43 that communicates with the information terminal device 1.

For example, a relationship graph based on time-series data of each attribute is not generated by each individual person, but a relationship graph is generated based on an average value of time-series data of each attribute of a large number of users. With this configuration, in a case where time-series data of attributes of a certain user are missing, it is possible to compensate for the missing by using an average relationship graph. Further, by comparing an average relationship graph with an individual relationship graph, it is possible to present a user that how far the user is away from a general person.

### [Configuration of Information Terminal Device 1]

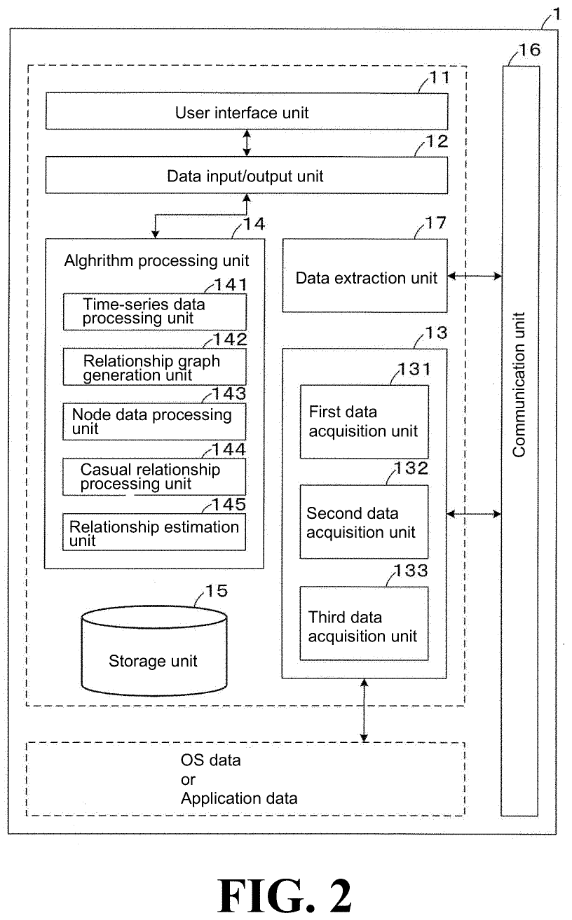

The information terminal device 1 is a device, such as, e.g., a smartphone, a tablet terminal, and a robotic device. As shown in FIG. 2, the information terminal device 1 is provided with a user interface unit 11 serving as an interface with a user, a data input/output unit 12 for inputting and outputting data, a data acquisition unit 13 for acquiring time-series data of each attribute, an algorithm processing unit 14 for processing a predetermined algorithm, a storage unit 15 for storing time-series data of each attribute and a relationship graph, a communication unit 16 for communicating with the outside via a network, and a data extraction unit 17 for extracting various types of data. Note that the information terminal device 1 stores and executes predetermined OS data and application data by a CPU or the like.

The user interface unit 11 is, for example, a display screen of a smartphone or the like. The user interface unit 11 has an input function for performing various operations by a user and an output function for presenting various types of information to the user.

The data input/output unit 12 transmits the input data input by the user interface unit 11 to the algorithm processing unit 14, and transmits the output data acquired from the algorithm processing unit 14 to the user interface unit 11.

The data acquisition unit 13 acquires time-series data of each attribute from outside the terminal device or inside the terminal device. The data acquisition unit 13 is provided with a first data acquisition unit 131, a second data acquisition unit 132, and a third data acquisition unit 133.

The first data acquisition unit 131 acquires time-series data of each attribute measured by the IoT device 2, such as, a wearable device. The second data acquisition unit 132 acquires time-series data of each attribute from the external Web service 3. Further, the third data acquisition unit 133 acquires time-series data of each attribute measured or input by an application of the information terminal device 1.

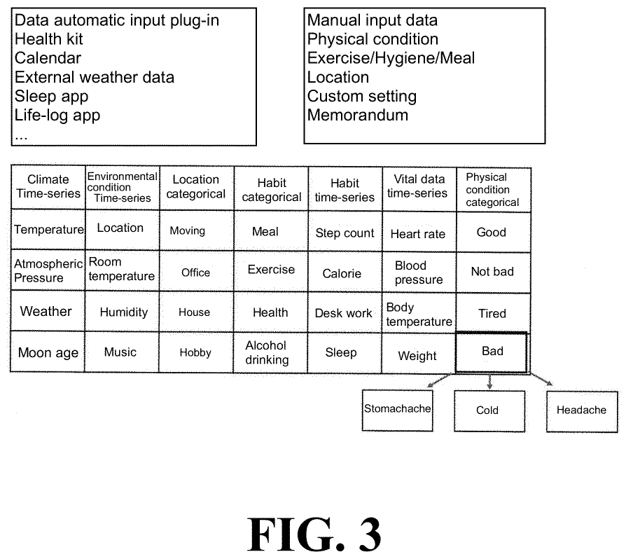

The time-series data of these attributes include time-series data of each attribute belonging to climatic time-series, environmental condition time-series, location categorical, habit categorical, habit time-series, vital data time-series, and physical condition categorical, as shown in FIG. 3. Among them, the time-series data of each attribute relating to the location categorical, the habit categorical, the habit time-series, the vital data time-series, and the physical condition categorical are often acquired from the IoT device 2 by the first acquiring unit. Further, the time-series data relating to attributes of climatic time-series and environmental condition time-series are often acquired from the external Web service 3 by the second data acquisition unit 132. Further, the time-series data of attributes relating to the vital data time-series are sometimes acquired by the third data acquisition unit 133 from the information terminal device 1. However, the above-described acquisition of the time-series data of each attribute is one example, and times-series data of other various types may be acquired.

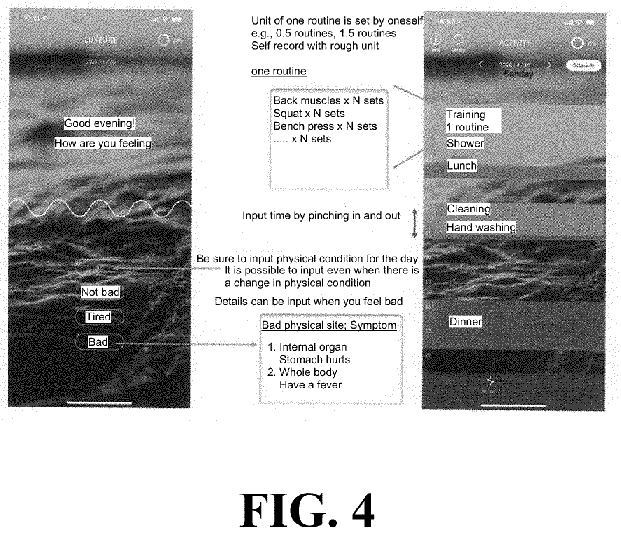

Further, in particular, the third data acquisition unit 133 automatically acquires time-series data of each attribute by a data auto-input plug-in in an application, such as, e.g., a health kit, a calendar, external weather data, a sleep app, and a life-log app installed on the information terminal device 1, and acquires time-series data of attributes relating to a physical condition, exercise, hygiene, meal, location, unique setting, notes, and the like by manual input. For example, as shown in FIG. 4, examples of acquiring time-series data of each attribute by manual input include: setting a routine unit by oneself and inputting a routine unit, such as, e.g., 0.5 routines or 1.5 routines; inputting a time of a habit, such as, e.g., lunch, cleaning, and hand washing; and inputting a physical condition of a day (when there is a bad physical condition, inputting the symptom).

Note that in this embodiment, as specific time-series data of attributes acquired by the data acquisition unit 13, the following can be exemplified.
asleep: Sleeping hours
temperature_min Minimum temperature on the day
Efficiency: Sleep Efficiency (Sleeping hours/Bedtime to Wake-up time)
wakingBPM_diff: Daily change (differential) in heart rate (Beats per minute) at wake-up
quality: Quality of sleeping
dayBPM: Daytime heart rate (Beats per minute)
wakingBPM: Heart rate at wake-up (Beats per minute)
pressure_noon_diff: Daily change (differential) in atmospheric pressure
hrv: Heart rate interval variation
deep: Hours of deep sleep
asleep_diff: Daily change (difference) in sleep duration

The algorithm processing unit 14 is provided with: a time-series data processing unit 141 for calculating similarity between time-series data of attributes; a relationship graph generation unit 142 for generating a relationship graph; a node data processing unit 143 for extracting an inter-node having a large total link weight in a relationship graph; a causal relationship processing unit 144 for calculating a causal relationship of an inter-node; and a relationship estimation unit 145 for estimating a relationship between attributes.

The time-series data processing unit 141 calculates the similarity between the time-series data of each attribute acquired by the data acquisition unit 13. In this embodiment, this time-series data processing unit 141 calculates the similarity between time-series data of each attribute by using DTW (Dynamic Time Warping). In the DTW, distances between points in two time-series are compared in a round-robin to find a path with the shortest distance between the time-series, and the shortest distance is a distance of the DTW. For this reason, even if the periodicity and/or the length of time-series data of two attributes are different, the DTW distance can be defined. The larger the similarity is, the shorter (closer) the distance is.

In this embodiment, the time-series data processing unit 141 normalizes the acquired time-series data of each attribute in advance. For example, in the case of temperature time-series data, it ranges from −5 degrees to 40 degrees, while in the case of daily step count time-series data, the absolute range varies greatly depending on time-series data, such as 0 to 20,000 steps. Therefore, the possible range of the absolute value differs largely, and therefore, it is normalized to a certain range (for example, a range of 0 to 1).

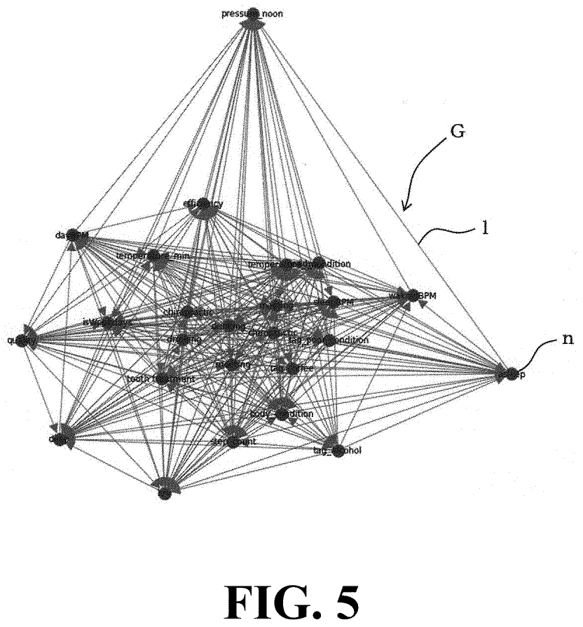

As shown in FIG. 5, the relationship graph generation unit 142 generates a non-directional relationship graph in which each attribute of time-series data acquired by the data acquisition unit 13 serves as a node, similarity between time-series data of each attribute calculated by the time-series data processing unit 141 serves as a direct link weight of an inter-node, and the nodes are connected by links. In FIG. 5, the reference symbol “G” represents a relationship graph, “l” represents a link, and “n” represents a node.

In this relationship graph, the higher the similarity between time-series data of attributes is, the larger the direct link weight of an inter-node corresponding to between attributes becomes. Further, the inter-node in which the similarity was calculated is directly connected by one link, while the inter-node in which similarity is not calculated is also indirectly connected by a plurality of links via other nodes. Therefore, each inter-node has a plurality of paths directly and indirectly connected by one or a plurality of links. The sum of link weights on a path constitutes the link weight of the path. The sum of link weights of a path of an inter-node constitutes a total link weight of the inter-node. In this embodiment, a link length is employed as a link weight of an inter-node, and the shorter the link length is, the larger the link weight is.

Note that in FIG. 5, the link of each inter-node in the relationship graph is oriented. However, after calculating the causal relationship of the inter-node by the causal relationship processing unit 144, which will be described later, the link is oriented by the node data processing unit 143, and therefore, the link has not been oriented when the relationship graph is initially generated.

The node data processing unit 143 calculates one or a plurality of paths (first to third paths in this embodiment) having a large link weight for each inter-node in the relationship graph generated by the relationship graph generation unit 142.

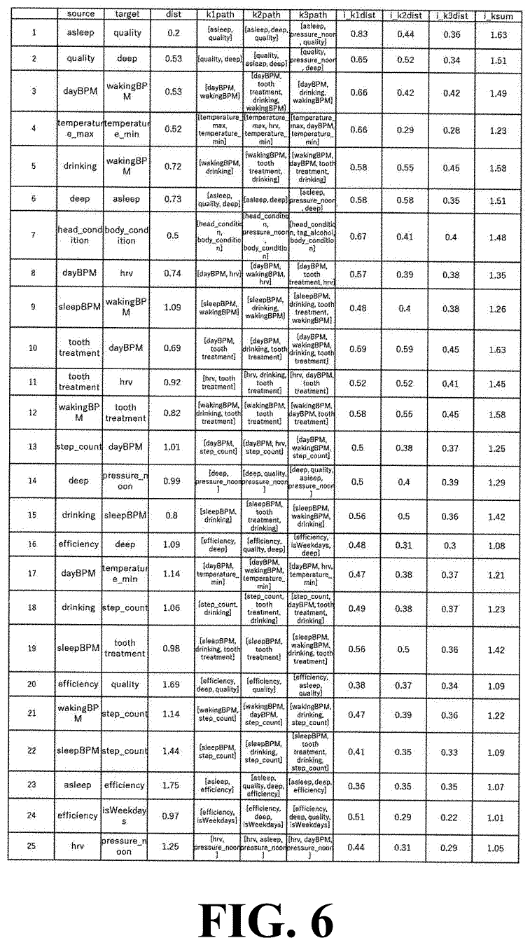

For example, FIG. 6 is a table showing a link weight of a path of each inter-node in a certain relationship graph. In FIG. 6, “source” denotes an original node (attribute), “target” denotes a node (source) which is a target of “source,” “dist” denotes a weight (link length) of a direct link between nodes, “k1path,” “k2path,” and “k3path” each denote a path having a large weight from the first to the third links between nodes (the first to third shortest paths), “i_k1dist,” “i_k2dist,” and “i_k3dist” each are a weight (inverse of the link length of the path) of a link of the first to third path of an inter-node, and “i_ksum” denotes a sum (the sum of the inverse number of the link length of the path: total score) of the link weights of the first to third paths of inter-nodes. The reason for taking the inverse of the length (path length) of the link of the path as a link weight of a path is as follows. That is, by adopting the inverse number, the shorter the path length becomes, the larger the total score becomes. With this, the larger the total score of a link weight of a path becomes, the more the path (short path) having a large link weight between nodes is, and therefore it is possible to evaluate that the total link weight is larger.

Further, the node data processing unit 143 extracts one or a plurality of inter-nodes having a large total link weight, based on one or a plurality of paths having a large link weight of a path calculated by the node data processing unit 143 for each inter-node. In this embodiment, the sum “i_ksum” of the link weights of the first to third paths calculated for each inter-node is a total link weight of the inter-node. Therefore, the node data processing unit 143 extracts an inter-node having a large total link weight. For example, according to FIG. 6, the sum “i_ksum” of the link weight of the path in the first row is large, and therefore, the inter-node of “asleep (sleeping hours)” and “quality (quality of sleep) in the first row is extracted as an inter-node having a large total link weight.

The causal relationship processing unit 144 calculates a causal relationship of an inter-node in a relationship graph. In this embodiment, the causal relationship processing unit 144 calculates a correlation (positive and negative polarities and a time gap) between time-series data of each attribute to assess the causal relationship of the inter-node in the relationship graph corresponding between time-series data of each attribute.

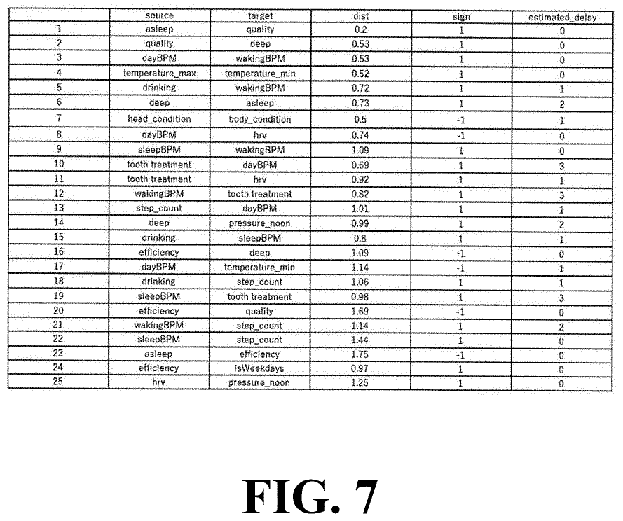

For example, FIG. 7 is a table showing the causal relationship between the attribute of “source” and the attribute of “target.” “Sign” is a numerical value representing the correlation between time-series data of an attribute of “source” and time-series data of an attribute of “target” “1” denotes a positive correlation, and “−1” denotes a negative correlation. In a case where an inter-attribute is a positive correlation, when one attribute becomes larger, the other attribute also becomes larger. On the other hand, in a case where an inter-attribute is a negative correlation, when one attribute becomes larger, the other attribute becomes smaller.

Further, in FIG. 7, “estimated_delay” represents a time gap between time-series data of each attribute. When the attribute of “target” is delayed with respect to “source,” the “source” becomes a factor. On the other hand, when the attribute of “source” is delayed with respect to “target,” “target” becomes a factor. For example, according to the first row of FIG. 7, the attribute of “asleep (sleeping hours)” has a positive correlation to the attribute of “quality (sleeping quality),” and the time gap is 0.

In calculating the correlation of time-series data of each attribute, as a method of calculating the correlation, a method is exemplified in which time-series data of an attribute of “source” and time-series data of an attribute of “target” are compared by writing them in a superimposed manner, in a superimposed manner with one of them inverted, or in a time-shifted manner.

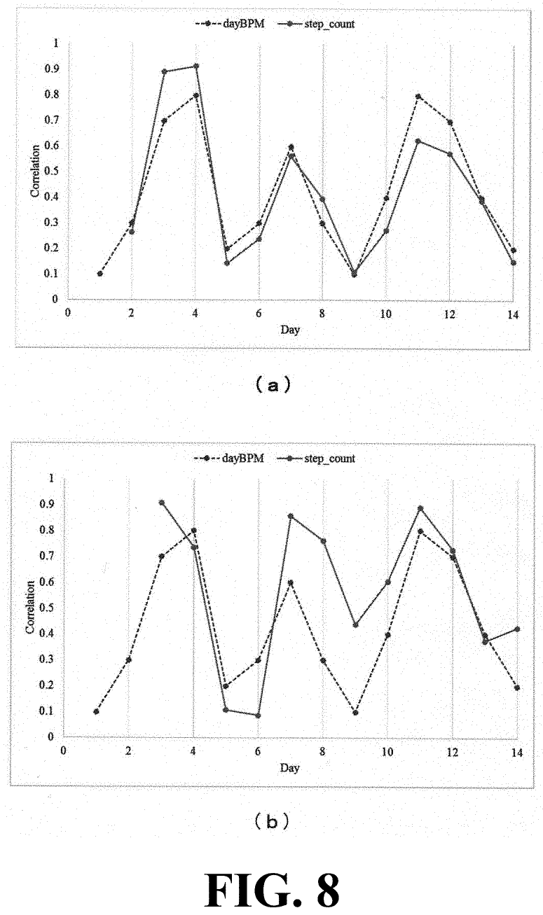

For example, FIG. 8 (a) shows time-series data of an attribute of “step_count (step count)” and time-series data of an attribute of “dayBPM (daytime heart rate)” by shifting the time-series data of the attribute of “step_count” by one day. According to FIG. 8 (a), the time-series data of the attribute of “step_count (step count)” and the time-series data of the attribute of “dayBPM (daytime hear rate)” are generally similar in the undulation. Therefore, there is a positive correlation between the attribute of “step_count (step count)” and the attribute of “dayBPM (daytime heart rate),” and it is understood that the attribute of “step_count (step count)” is a factor of the attribute of “dayBPM (daytime heart rate)” after one day.

On the other hand, FIG. 8 (b) similarly shows time-series data of an attribute of “step_count (step count)” and the time-series data of an attribute of “dayBPM” (daytime heart rate) by inverting the time-series data of the attribute of “step_count (step count) and then shifting the data by two days toward future. According to FIG. 8 (b), the time-series data of the attribute of “step_count (step count)” and the time-series data of the attribute of “dayBPM (daytime heart rate)” coincide in the undulation in some points. However, as compared with FIG. 8 (a), the correlation is considered to be lower. For this reason, the correlation (positive correlation, one day gap) according to FIG. 8 (a) is adopted.

Note that the causal relationship processing unit 144 is not necessarily required to calculate causal relationships of all of inter-nodes, and may calculate, for example, only causal relationships of inter-nodes having a large total link weight extracted by the node data processing unit 143.

Thus, as shown in FIG. 5, the node data processing unit 143 gives an orientation (the origin of the arrow denotes a factor/the tip of the arrow is a result) to a link of each inter-node, based on the causal relationship of the inter-node in the relationship graph calculated by the causal relationship processing unit 144.

The relationship estimation unit 145 estimates the relationship between the inter-attribute corresponding to the inter-node extracted by the node data processing unit 143. For example, in a case where an inter-node of “asleep (sleeping hours)” and “quality (sleep quality)” are extracted by the node data processing unit 143, the relationship estimation unit 145 estimates that there is an inter-attribute relationship of “asleep (sleeping hours)” and quality (sleep quality)” or there is a strong inter-attribute relationship between “asleep (sleeping hours)” and “quality (sleep quality).” At this time, in a case where, in the relationship graph, an arrow (“asleep (sleeping hours)” is located at the origin of the arrow, and “quality (sleep quality)” is located at the tip of the arrow) have been given to the direct link of the inter-node of “asleep (sleeping hours)” and “quality (sleep quality),” it is estimated that “asleep (sleeping hours)” affects “quality (sleep quality).” Further, in a case where the “estimated_day” (time gap) of “asleep (sleeping hours)” and “quality (quality of sleep)” are calculated as one day by the causal relationship processing unit 144, it is estimated that “quality (quality of sleep)” is influenced after one day of “asleep (sleeping hours).”

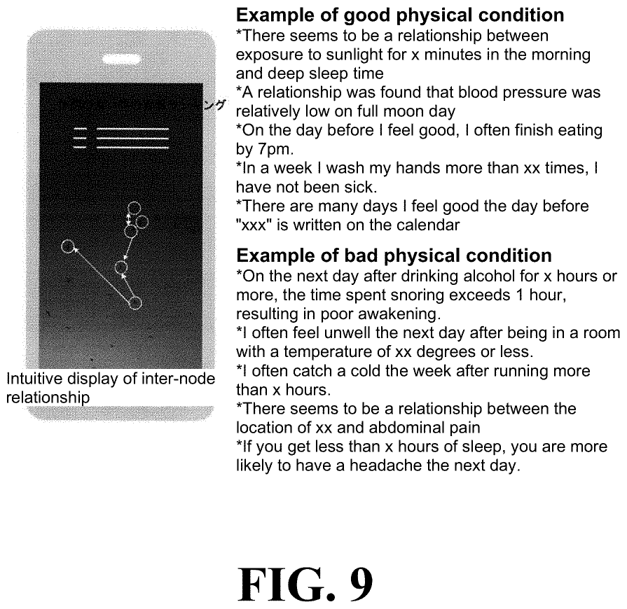

In addition, when estimating the relationship between attributes, as shown in FIG. 9, the relationship estimation unit 145 outputs the relationship between attributes as a sentence (see the right side of FIG. 9) on the screen of the user interface unit 11, or outputs the relationship as an intuitive graph (see the inside of the screen on the left side of FIG. 9).

The data extraction unit 17 transmits the time-series data and/or the relationship graph of each attribute to the server device 4 via the communication unit 16, extracts various information received from the server device 4 via the communication unit 16, and transmits the information to the algorithm processing unit 14.

[Operation of this System]

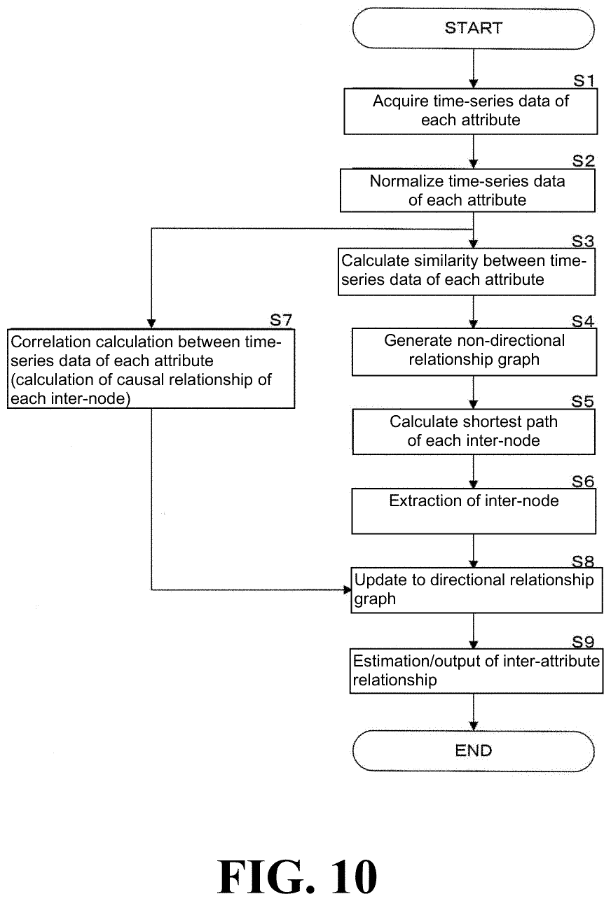

Next, the operation of this system will be described below with reference to the flowchart shown in FIG. 10.

First, the data acquisition unit 13 (the first to third data acquisition units 131 to 133) acquires time-series data of a plurality of types of attributes from the IoT device 2 outside the terminal device, the external Web service 3, or an application inside the terminal device (S1).

Then, the time-series data processing unit 141 normalizes the time-series data of each attribute acquired by the data acquisition unit 13 in advance (S2), and then calculates the similarity between the time-series data of each attribute (S3).

Then, the relationship graph generation unit 142 generates a non-directional relationship graph in which nodes are connected with links by using each attribute of the time-series data as a node and the similarity of time-series data of each attribute calculated by the time-series data processing unit 141 as a link weight of each inter-node (S4).

Then, the node data processing unit 143 calculates one or a plurality of paths (shortest path) having a large weight link for each inter-node, in the relationship graph generated by the relationship graph generation unit 142 (S5).

Then, the node data processing unit 143 extracts one or a plurality of inter-nodes having a large total link weight, based on one or a plurality of paths having a large link weight of a path calculated for each link (S6).

On the other hand, the causal relationship processing unit 144 calculates the causal relationship of each inter-node of the relationship graph corresponding to each inter-attribute by calculating the correlation between time-series data of each attribute (S7).

Then, as shown in FIG. 5, the node data processing unit 143 updates the relationship graph to a directional relationship graph by giving the orientation (the origin of the arrow is the factor/the tip of the arrow is the result) to the link of each inter-node of the relationship graph, based on the causal relationship of each inter-node in the relationship graph calculated by the causal relationship processing unit 144 (S8).

The relationship estimation unit 145 estimates the relationship of the inter-attribute corresponding to the inter-node extracted by the node data processing unit 143. At this time, the relationship estimation unit 145 may estimate the relationship between attributes based on the orientation of the link given to the relationship graph, or may directly estimate the relationship between attributes based on the causal relationship (the factor and the result, the time gap) of the inter-node calculated by the causal relationship processing unit 144. Further, the relationship estimation unit 145 outputs the relationship between attributes to the screen of the interface as a sentence (on the right side of FIG. 9) or as an intuitive graph (within the screen on the left side of FIG. 9) (S9).

Note that in this embodiment, the causal relationship processing unit 144 for calculating the causal relationship of the inter-node is provided, but the causal relationship processing unit 144 may not be provided. In this instance, the relationship estimation unit 145 only estimates that there is some relationship of the inter-attribute when estimating the relationship of the inter-attribute of the inter-node extracted by the node data processing unit 143.

Further, the node data processing unit 143 gives the orientation to the link of the inter-node of the relationship graph based on the causal relationship of the inter-node calculated by the causal relationship processing unit 144, but it may be configured such that it may not give the orientation of the link to the inter-node of the relationship graph. In this instance, the relationship estimation unit 145 may be configured to directly estimate the relationship of the inter-attribute corresponding to the inter-node, based on the causal relationship of the inter-node calculated by the causal relationship processing unit 144.

Further, the relationship graph generation unit 142 uses the similarity between time-series data of an attribute calculated by the time-series data processing unit 141 as the link weight of the inter-node. However, the similarity between the time-series data of the attribute may be used to calculate the link weight of the inter-node by other methods. For example, the similarity between time-series data of an attribute is used as a weight of a node itself, and the node itself may be used to define the weight.

### EXAMPLES

### Example 1

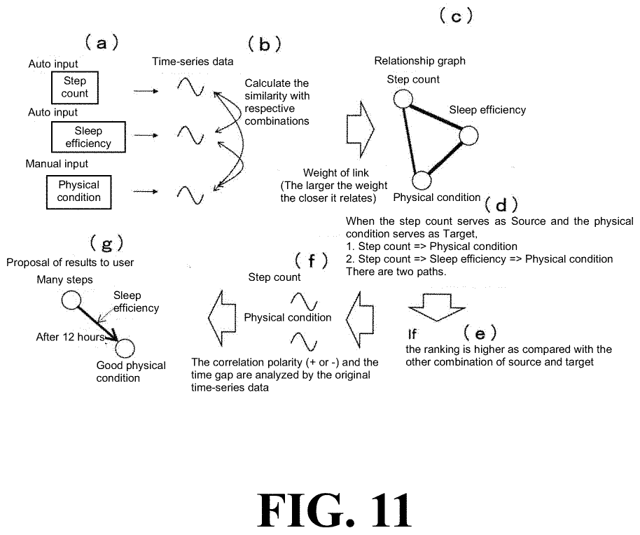

Next, Example 1 of the present invention will be described with reference to FIG. 11.

In this Example 1, the following description is directed to the case in which as time-series data of attributes, time-series data of a step count, and time-series data of sleep efficiency are automatically acquired from the IoT device 2, and time-series data of a physical condition are manually acquired from the information terminal device 1, but, in actual, time-series data of other attributes are also acquired.

First, as shown in FIG. 11 (a), the data acquisition unit 13 (the first to third data acquisition units 131 to 133) acquires time-series data of a plurality of types of attributes including time-series data of the above-described three types of attributes (step count, sleep efficiency, and physical condition) from the IoT device 2 outside the terminal device or the external Web service 3 or an application inside the terminal device.

As shown in FIG. 11 (b), the time-series data processing unit 141 normalizes the time-series data of each attribute acquired by the data acquisition unit 13 in advance and then calculates the similarity between the time-series data of each attribute, such as, e.g., a step count, sleeping efficiency, and a physical condition.

Then, as shown in FIG. 11 (c), the relationship graph generation unit 142 generates a non-directional relationship graph in which nodes are connected by links with each attribute (a step count, sleep efficiency, a physical condition, and the like) of each attribute of each time-series data serving as a node, and with the similarity of the time-series data of each attribute (a step count, sleep efficiency, a physical condition, and the like) calculated by the time-series data processing unit 141 serving as the link weight of each inter-node. Note that in FIG. 11 (c), a relationship graph in which three nodes of the step count, the sleep efficiency, and the physical condition are connected by links is shown. However, in actual, a relationship graph in which other nodes are also connected by links is generated.

As shown in FIG. 11 (d), the node data processing unit 143 calculates one or a plurality of paths of inter-nodes having a large link weight in the relationship graph generated by the relationship graph generation unit 142. For example, in a case where the step count serves as a node (attribute) of “Source,” and the physical condition serves as a node (attribute) of “Target,” there are two paths, i.e., a path (step count-physical condition) and a path (step count-sleep efficiency-physical condition). Therefore, these two paths are calculated, and a total link weight of the inter-node of the step count and the physical condition is calculated based on the link weights of the two paths.

Then, as shown in FIG. 11 (e), in a case where the total link weight of the inter-node of the step count and the physical condition is higher as compared with the other combinations of a node of “Source” and a node of “Target,” the node data processing unit 143 extracts the inter-node of the step count and the physical condition.

Then, as shown in FIG. 11 (f), the causal relationship processing unit 144 calculates the correlation (positive and negative polarities, time gap) between time-series data of attributes (step count, physical condition) to assess the causal relationship (the step count is a cause, the physical condition is a result, a time gap of 12 hours) of the inter-node in the relationship graph corresponding to between time-series data of the attribute.

Then, as shown in FIG. 11 (g), the node data processing unit 143 gives the orientation (step count→physical condition) to the link of the inter-node of the step count and the physical condition in the relationship graph, based on the causal relationship (the step count is a factor, and the physical condition is a result) of the inter-node in the relationship graph calculated by the causal relationship processing unit 144.

Further, as shown in FIG. 11 (g), the relationship estimation unit 145 estimates that the relationship between the step count and the physical condition is strong, that the step count affects the physical condition after 12 hours, and that the sleep efficiency relates between the step count and the physical condition, and outputs a predetermined sentence or graph on the screen of the user interface unit 11.

### Example 2

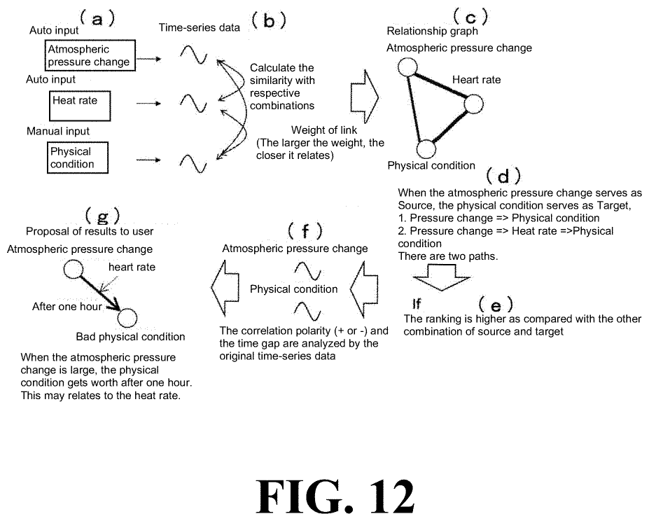

Next, Example 2 of the present invention will be described with reference to FIG. 12.

In Example 2, the following description is directed to the case in which, as time-series data of attributes, time-series data of atmospheric pressure change automatically acquired from the IoT device 2, time-series data of the heart rate automatically acquired from the information terminal device 1, and time-series data of physical condition manually acquired from the information terminal device 1 are acquired, however, in actual, time-series data of other attributes are also acquired.

First, as shown in FIG. 12 (a), the data acquisition unit 13 (the first to third data acquisition units 131 to 133) acquires time-series data of a plurality of types of attributes including times-series data of the above-described three types of attributes (atmospheric pressure change, heart rate, physical condition) from the IoT device 2 outside the terminal device, the external Web service 3, or an application inside the terminal device.

As shown in FIG. 12 (b), the time-series data processing unit 141 normalizes the time-series data of each attribute acquired by the data acquisition unit 13 in advance, and then calculates the similarity between the time-series data of each attribute, such as the atmospheric pressure change, the heart rate, and the physical condition.

Then, as shown in FIG. 12 (c), the relationship graph generation unit 142 generates a non-directional relationship graph in which nodes are connected by links with each attribute (atmospheric pressure change, heart rate, physical condition, etc.) of each time-series data serving as a node and with the similarity of the time-series data of each attribute (atmospheric pressure change, heart rate, physical condition, etc.) calculated by the time-series data processing unit 141 serving as a link weight of each inter-node. Note that in FIG. 12 (c), a relationship graph in which three nodes of the pressure change, the heart rate, and the physical condition are connected by links is shown. However, in actual, a relationship graph in which other nodes are connected by links is generated.

As shown in FIG. 12 (d), the node data processing unit 143 calculates one or a plurality of paths of inter-nodes having a large link weight, in the relationship graph generated by the relationship graph generation unit 142. For example, in a case where the atmospheric pressure change serves as a node (attribute) of “Source,” and the physical condition serves as a node (attribute) of “Target,” there are two paths, i.e., a path (atmospheric pressure change-physical condition) and a path (atmospheric pressure change-heart rate-physical condition). Therefore, these two paths are calculated, and a total link weight of the inter-node of the atmospheric pressure change and the physical condition is calculated based on the link weight of the two paths.

Then, as shown in FIG. 12 (e), in a case where the total link weight of the inter-node of the atmospheric pressure changes and the physical condition is higher as compared with the other combinations of a node of “Source” and a node of “Target,” the node data processing unit 143 extracts the inter-node of the atmospheric pressure change and the physical condition.

Then, as shown in FIG. 12 (f), the causal relationship processing unit 144 calculates the correlation (positive and negative polarities, time gap) between time-series data of attributes (step count, physical condition) to acquire the causal relationship (the step count is a cause, the physical condition is a result, a time gap of 1 hour) of the inter-node in the relationship graph corresponding to between time-series data of the attribute (atmospheric pressure change, the physical condition).

Then, as shown in FIG. 12 (g), the node data processing unit 143 gives the orientation (atmospheric pressure change→physical condition) to the link of the inter-node of the atmospheric pressure change and the physical condition) in the relationship graph, based on the causal relationship (the atmospheric pressure change is a factor, and the physical condition is a result) of the inter-node in the relationship graph calculated by the causal relationship processing unit 144.

Further, as shown in FIG. 12 (g), the relationship estimation unit 145 estimates that the relationship between the atmospheric pressure change and the physical condition is strong, that the atmospheric pressure change affects the physical condition after 1 hours, and that the heart rate relates between the atmospheric pressure change and the physical condition, and outputs a predetermined sentence or graph on the screen of the user interface unit 11.

### Example 3

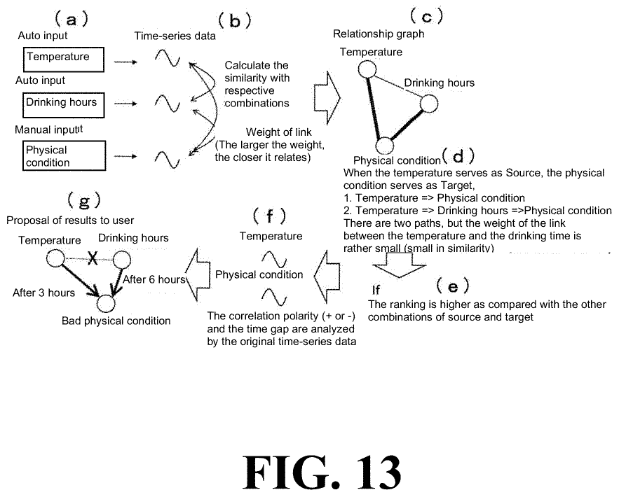

Next, Example 3 of the present invention will be described with reference to FIG. 13.

In this Example 3, the following description is directed to the case in which, as time-series data of attributes, time-series data of a temperature are automatically acquired from the IoT device 2, and time-series data of the drinking hours and the time-series data of the physical condition are manually acquired from the information terminal device 1, but, in actual, time-series data of other attributes are also acquired.

First, as shown in FIG. 13 (a), the data acquisition unit 13 (the first to third data acquisition units 131 to 133) acquires time-series data of a plurality of types of attributes including times-series data of the above-described three types of attributes (temperature, drinking hours, physical condition) from the IoT device 2 outside the terminal device, the external Web service 3, or an application inside the terminal device.

Then, as shown in FIG. 13 (b), the time-series data processing unit 141 normalizes the time-series data of each attribute acquired by the data acquisition unit 13 in advance, and then calculates the similarity between the time-series data of each attribute, such as the temperature, the drinking hours, and the physical condition.

Then, as shown in FIG. 13 (c), the relationship graph generation unit 142 generates a non-directional relationship graph in which nodes are connected by links with each attribute (temperature, drinking hours, physical condition, etc.) of each time-series data serving as a node and with the similarity of the time-series data of each attribute (temperature, drinking hours, physical condition, etc.) calculated by the time-series data processing unit 141 serving as a link weight of each inter-node. Note that in FIG. 13 (c), a relationship graph in which three nodes of the temperature, the drinking hours, and the physical condition are connected by links is shown. However, in actual, a relationship graph in which other nodes are connected by links is generated.

As shown in FIG. 13 (d), the node data processing unit 143 calculates one or a plurality of paths of inter-nodes having a large link weight, in the relationship graph generated by the relationship graph generation unit 142. For example, in a case where the temperature serves as a node (attribute) of “Source,” and the physical condition serves as a node (attribute) of “Target,” there are two paths, i.e., a path (temperature-physical condition) and a path (temperature-drinking hours-physical condition). Therefore, these two paths are calculated, and a total link weight of the inter-node of the temperature and the physical condition is calculated based on the link weight of the two paths. Note that in the path (temperature-drinking hours-physical condition), it is assumed that the link weight between the node (drinking hours) and the node (physical condition) is small.

Then, as shown in FIG. 13 (e), in a case where the total link weight of the inter-node of the temperature and the physical condition is higher as compared with the other combinations of a node of “Source” and a node of “Target,” the node data processing unit 143 extracts the inter-node of the temperature and the physical condition.

Then, as shown in FIG. 13 (f), the causal relationship processing unit 144 calculates the correlation (positive and negative polarities, time gap) between time-series data of attributes (temperature, physical condition) to acquire the causal relationship (the temperature is a cause, the physical condition is a result, a time gap of 3 hours) of the inter-node in the relationship graph corresponding to between time-series data of the attribute (temperature, the physical condition).

Then, as shown in FIG. 13 (g), the node data processing unit 143 gives the orientation (temperature→physical condition) to the link of the inter-node of the temperature and the physical condition in the relationship graph, based on the causal relationship (the temperature is a factor and the physical condition is a result) of the inter-node in the relationship graph calculated by the causal relationship processing unit 144. Note that in a case where the node data processing unit 143 has extracted the inter-node between the drinking hours and the physical condition as an inter-node having a large total link weight, the node data processing unit 143 gives the orientation (drinking hours→physical condition) to the link of the inter-node of the drinking hours and the physical condition, based on the causal relationship (the drinking hours is a factor, and the physical condition is a result) of the inter-node in the relationship graph calculated by the causal relationship processing unit 144.

Further, as shown in FIG. 13 (g), the relationship estimation unit 145 estimates that the relationship between the temperature and the physical condition is strong, the temperature affects the physical condition after 3 hours, the relationship between the drinking hours and the physical condition is strong, the drinking hours affect the physical condition after 6 hours, and the temperature and the drinking hours is weak in the relationship, and outputs a predetermined sentence or graph on the screen of the user interface unit 11.

The embodiments of the present invention have been described above with reference to the attached drawings, but the present invention is not limited to the illustrated embodiments. It should be understood that various modifications and variations can be made to the illustrated embodiments falling within the same or equivalent scope as the present invention.

### DESCRIPTION OF SYMBOLS

1: User information terminal device
   11: User interface unit
   12: Data input/output unit
   13: Data acquisition unit
      131: First data acquisition unit
      132: Second data acquisition unit
      133: Third data acquisition unit
   14: Algorithm processing unit
      141: Time-series data processing unit
      142: Relationship graph generation unit
      143: Node data processing unit
      144: Causal relationship processing unit
      145: Relationship estimation unit
   15: Storage unit
   16: Communication unit
   17: Data extraction unit
2: IoT device
   21: Sensor unit
   22: Communication unit
3: External Web service
4: Server Device
   41: Learning unit
   42: Storage unit
   43: Communication unit

1. A relationship estimation system comprising:
a data acquisition unit configured to acquire time-series data of a plurality of attributes, the time-series data including at least time-series data of attributes relating to a body or activities of a person;
a time-series data processing unit configured to calculate similarity between the time-series data of the attributes acquired by the data acquisition unit;
a relationship graph generation unit configured to generate a relationship graph in which the attributes of the time-series data acquired by the data acquisition unit serve as nodes and the nodes are connected by links, the relationship graph generation unit being configured to utilize the similarity between the time-series data of the attributes calculated by the time-series data processing unit to calculate a link weight of an inter-node;
a node data processing unit configured to extract the inter-node having a large total link weight in the relationship graph generated by the relationship graph generation unit; and
a relationship estimation unit configured to estimate a relationship between the attributes corresponding to the inter-node extracted by the node data processing unit.

2. The relationship estimation system as recited in claim 1,
wherein the relationship graph generation unit generates the relationship graph by using the similarity between the time-series data of the attributes calculated by the time-series data processing unit as the link weight of the inter-node.

3. The relationship estimation system as recited in claim 1,
wherein the node data processing unit calculates a plurality of paths having a large link weight for each inter-node in the relationship graph generated by the relationship graph generation unit and extract the inter-node having a large total link weight based on the link weight of each path.

4. The relationship estimation system as recited in claim 3,
wherein the node data processing unit calculates a total link weight of the inter-node by computing the link weight for each path of the inter-node.

5. The relationship estimation system as recited in claim 4,
wherein the node data processing unit calculates the total link weight of the inter-node by adding an inverse of a link length for each path of the inter-node as a link weight.

6. The relationship estimation system as recited in claim 1, further comprising:
a causal relationship processing unit configured to assess a causal relationship of the inter-node in the relationship graph by calculating a correlation between the time-series data of the attributes.

7. The relationship estimation system as recited in claim 6,
wherein the causal relationship processing unit calculates positive and negative polarities and a time gap of the correlation between the time-series data of the attributes.

8. The relationship estimation system as recited in claim 6,
wherein the node data processing unit orients a link of the inter-node in the relationship graph based on the causal relationship of the inter-node assessed by the causal relationship processing unit.

9. The relationship estimation system as recited in claim 6,
wherein the relationship estimation unit estimates the relationship between the attributes corresponding to the inter-node, based on the causal relationship of the inter-node assessed by the causal relationship processing unit.

10. The relationship estimation system as recited in claim 1,
wherein the data acquisition unit acquires the time-series data including at least one of time-series data of attributes measured by a sensor of an external IoT device, time-series data of attributes provided by an external Web service, and a time-series data of attributes measured or input by an information terminal device mounting the relationship estimation system.

11. The relationship estimation system as recited in claim 1,
wherein the time-series data processing unit normalizes the time-series data of each of the attributes acquired by the data acquisition unit.

12. An information terminal device equipped with the relationship estimation system as recited in claim 1.

13. A relationship estimation system comprising:
a plurality of information terminal devices as recited in claim 12; and
a server device connected to each of the information terminal devices via a network,
wherein the server device generates a relationship graph based on time-series data of attributes collected from each of the information terminal devices.
